(19) United States
(12) Patent Application Publication
(10) Pub. No.: US 2025/0354971 A1
(43) Pub. Date: Nov. 20, 2025

(54) METHODS FOR EVALUATING THE IMPACT OF CHEMICAL PRODUCTS ON PRODUCED WATER

(71) Applicants: PETROLEO BRASILEIRO S.A. - PETROBRAS, Rio de Janeiro (BR); SERVICO NACIONAL DE APRENDIZAGEM INDUSTRIAL - SENAI RJ, Rio de Janeiro (BR)

(72) Inventors: Rogerio Mesquita De Carvalho, Rio de Janeiro (BR); Antonio Augusto Fidalgo Neto, Rio de Janeiro (BR); Rafaella Magliano Balbi De Faria, Rio de Janeiro (BR); Aline Machado De Azevedo Novaes, Rio de Janeiro (BR); Sirlene Oliveira Francisco De Azeredo, Rio de Janeiro (BR)

(21) Appl. No.: 19/206,604

(22) Filed: May 13, 2025

(30) Foreign Application Priority Data
May 15, 2024 (BR) 1020240096207

(51) Int. Cl. G01N 33/18; G01N 5/04

(52) U.S. Cl. CPC G01N 33/18; G01N 5/04

(57) ABSTRACT

The present invention refers to a method for evaluating the impact of chemical products on the OGC of produced water, comprising the steps of: (i) preparation of synthetic emulsions using oil mimicking produced water, produced water mimicked with the chemical product whose impact is to be evaluated, and saline water with said chemical product (without oil); (ii) measurement of the OGC of the samples; and (iii) evaluation of the obtained data.

Further, the invention refers to a method for evaluating the impact of chemical products on the reinjection of produced water, comprising the steps of: (I) preparation of synthetic emulsions using oil mimicking produced water and produced water mimicked with the chemical product whose impact is to be evaluated; (II) measurement of the OGC of the samples; (III) measurement of the filtration time; and (IV) evaluation of the obtained data.

### FIELD OF THE INVENTION

The present invention is part of the field of petroleum processing, more precisely for quality control of the produced water, and describes methods for evaluating the impact of the chemical products used in the petroleum processing on the produced water, particularly on the oil and grease content, and its suitability for disposal and/or reinjection.

### BACKGROUNDS OF THE INVENTION

The use of various chemical products is common in primary petroleum processing. For example, demulsifiers, antifoams, scale and corrosion inhibitors and H₂S scavengers are added to the oil stream. As for the produced water, separated from the oil, biocides, polyelectrolytes, O₂ scavengers, among others, are added, depending on the operation.

When the products have some type of surfactant activity, regardless of where they are added, they can result in an increase in the concentration of oil dispersed in the water, which can result in a serious problem. The discharge of produced water must comply with the limits established in CONAMA 393/07, with the oil concentration (Oil and Grease Content—OGC) being a maximum of 29 mg/L per month and 42 mg/L per day.

Depending on the product used, mainly in situations involving a change in chemical product or an increase in the concentration used, an increase in the OGC may occur, resulting in fines imposed by the environmental agencies, in compliance with the legislation in place.

In turn, when the produced water is reinjected into the reservoir, the increase in the OGC value may lead to damage to the reservoir, specifically with an increase in the speed at which a type of cake is formed, which plugs the rock and results in an increase in the injection pressure so that the flow rate is maintained. However, there is a limit that cannot be exceeded regarding the rate of pressure increase, with the risk of fracturing and other possible undesirable consequences (e.g., oil exudation).

Additionally, as damage develops, the frequency of interventions to remove the same increases, resulting in higher annual operating costs.

In general, the impact of products on OGC is an item that should be made available by the chemical product suppliers, but this information is often not available, and often such an evaluation is not even carried out.

Another approach is to carry out tests at the primary processing plant itself with several active bases, as a way of directly evaluating the impact. However, for offshore production systems, there is a need for boarding to carry out this specific activity, the complexity of which is greatly influenced by the type of product to be evaluated. For example, for H₂S scavengers, the evaluation is restricted to their efficiency in reducing the concentration of H₂S present, with other not aspects being evaluated concomitantly.

Therefore, there is a need in the art to provide information about the impact of the use of chemical products on the produced water, mainly when a new product starts to be used.

### STATE OF THE ART

Some documents of the state of the art address to the determination of OGC of produced water, using emulsions that mimic produced water, such as:

The document titled “DETERMINAÇÃO E CORRELAÇÃO DO TEOR TOTAL DE ÓLEOS E GRAXAS POR MEIO DE DIFERENTES TÉCNICAS ESPECTROSCÓPICAS E GRAVIMÉTRICA”(“DETERMINATION AND CORRELATION OF THE TOTAL CONTENT OF OILS AND GREASES BY MEANS OF DIFFERENT SPECTROSCOPIC AND GRAVIMETRIC TECHNIQUES”) reports a comparative study of different methods of evaluating OGC in an oily water sample. The oily water samples were prepared synthetically from the addition of petroleum to a brine with a concentration of 55, 000 ppm (containing NaCl and CaCl₂ at 10:1), with subsequent addition of more saline solution.

Regarding the gravimetric analysis, said paper discloses a process for determining the OGC of the synthetic samples comprising three extractions with n-hexane, drainage of the organic phase into a funnel containing filter paper and anhydrous sodium sulfate, and the organic phase being collected in an Erlenmeyer flask, which is subsequently subjected to a process of magnetic stirring with silica gel, filtration, rotary evaporation of the solvent and resting of the dry extract under a nitrogen atmosphere.

However, unlike the present invention, the document mentioned above discloses the preparation of mimetic emulsions of produced water with a known OGC content and without the elimination of volatiles. Furthermore, the emulsions are prepared with the aid of ultrasound, which aims at dispersing the oil in small droplets to keep the emulsion stable for as long as possible.

In the present invention, the objective of the developed method is different from that disclosed in the aforementioned document, being aimed at evaluating the impact of chemical products on OGC and not at comparing different methods of determining OGC. For this purpose, it is necessary to prepare emulsions with a known concentration of oil. Thus, in the invention, there is a variation in the used oil, as well as in the amount of oil and the dispersion form used in the preparation of the emulsion, in addition to a form of comparative evaluation, considering the absence and the presence of the chemical product targeted by the test, which is not provided for in the document of the state of the art referenced above.

In turn, the dissertation titled “TRATAMENTO DE ÁGUA PRODUZIDA UTILIZANDO OS PROCESSOS DE FLOTAÇÃO, OZONIZAÇÃO E SEPARAÇÃO POR MEMBRANAS” (“TREATMENT OF PRODUCED WATER USING FLOTATION, OZONIZATION AND MEMBRANE SEPARATION PROCESSES”) discloses the use of synthetic emulsions to evaluate the effects of changes in the petroleum extraction process on the OGC indices of the produced water. The dissertation reports the evaluation of the effectiveness of a treatment process of the produced water using synthetic emulsions as a base.

The mentioned document discloses the preparation of different emulsions, namely: oil and water; oil-salt-water (petroleum added to water with NaCl); oil-viscosifier-water (viscosifier added to the oil and water emulsion); oil-surfactant-water (emulsifier added to the oil and water emulsion); oil-surfactant-viscosifier-water (emulsifier and surfactant added to the oil and water emulsion); and oil-viscosifier-surfactant-salt-water (emulsifier and surfactant added to the oil-salt-water emulsion).

Unlike the present invention, the process reported in the dissertation mentioned above seeks to evaluate the efficiency in reducing OGC of treatment processes of the produced water. Furthermore, the preparation of the emulsions itself is more elaborate, requiring the use of a heated tank with mechanical stirring. The emulsions of the method of the present invention are simpler and can be prepared on a smaller scale.

Furthermore, the objective of the method of the present invention is different from that disclosed in the aforementioned dissertation, being aimed at evaluating the impact of chemical products on the OGC and not at evaluating the efficiency of produced water treatment methods. In the present invention, there is a variation in the type and quantity of petroleum used in the preparation of the emulsion, which is not provided for in the document of the state of the art referenced above. Furthermore, in the present invention, the preparation of the dispersion is carried out in such a way as to simulate a separation vessel interface and enable the comparison of the migration of oil to the aqueous phase in the absence and presence of the target chemical product of the test.

In turn, the document titled “Protocol for Preparing Synthetic Solutions Mimicking Produced Water from Oil and Gas Operations” refers to synthetic emulsions that mimic produced water. In particular, the emulsions disclosed in the aforementioned document comprise a brine (500 mL), a petroleum source (0.18 mL) and a surfactant (30 mg). The concentration of the brine (4 g/L or 40 g/L) and of the crude oil/petroleum added varies according to the produced water that one wishes to mimic. The document further teaches that synthetic emulsions can be used to test membranes useful in the oil and gas industry.

As is the case with the other documents mentioned herein, this scientific paper discloses the preparation of synthetic emulsions with a known OGC concentration. In addition, the emulsion whose preparation is reported in this paper is stable due to the presence of the surfactant used, which is absent in the synthetic emulsions of the present invention. In the emulsions of the present invention, the OGC value resulting from the preparation varies according to the characteristics of the oil and water used in the preparation, being a differential. The concentrations of salts added to the aqueous phase are also different when the synthetic emulsion of the aforementioned scientific paper is compared with the present invention.

The document also does not address to processes for evaluating the impact of different chemical products on the OGC, which is the objective of the present invention. Analogously to other documents of the state of the art, the objective is to prepare emulsions with known OGC and not to evaluate the impact of chemical products on the OGC.

The thesis titled “DESENVOLVIMENTO DE PADRÕES DE ÓLEOS E GRAXAS PARA DETERMINAÇÃO DE ÓLEOS E GRAXAS TOTAIS (TOG) EM ÁGUA PRODUZIDA: Avaliação de métodos alternativos para aplicação offshore” (“DEVELOPMENT OF OIL AND GREASE STANDARDS FOR DETERMINING TOTAL OIL AND GREASE (OGC) IN PRODUCED WATER: Evaluation of alternative methods for offshore application”) discloses a process for preparing mimetic emulsions of produced water comprising oil dispersed in an aqueous solution of NaCl 3.5% (m/v). Furthermore, the document discloses a process for evaluating OGC by gravimetry, in which samples previously acidified with HCl 37% to pH 2 are subjected to 3 extractions with hexane, followed by filtration in a bed of anhydrous sodium sulfate and transferred to a container for evaporation of the solvent and determination of the weight of the dry extract.

Furthermore, the thesis mentions other ways of preparing emulsions, namely: (i) preparation in large volumes for subsequent fractionation, in which known masses (20 to 100 mg/L) were weighed and dispersed in a volume of 8 L of water, using an ultra-turrax mixer (salinity 35 g/L and pH equal to 2); (ii) preparation with direct weighing, in which the known masses of oil are added directly to volumes of 1 L of water; and (iii) preparation with indirect weighing, in which the desired mass of oil is weighed in a crucible containing 1 g of sodium chloride and the mixture transferred to another container containing 900 mL of water (35 g/L of salinity and pH equal to 2). It is clear that the procedure focuses on the preparation of standard solutions.

As is the case with the documents discussed above, this thesis also aims at preparing emulsions with known concentrations from the dispersion of oils, in this specific case, volatile-free oil. In particular, the objective of this thesis is to prepare highly reliable OGC standards. In other words, the objective differs from that of the present invention, in which the OGC is unknown and variable, depending on the chemical product whose impact is to be evaluated and the composition of the oil tested itself, which must be equivalent to the petroleum to be processed with the product to be evaluated.

The doctoral thesis describes the use of these various standards in the validation of different OGC measurement methods. There is no correlation with the simulation of what occurs at a separator vessel interface, the effect of the chemical product on oil carryover and increase in OGC or the impact of the chemical product on reinjection.

Unlike all the documents mentioned above, the present invention prepares a mixture with a high quantity of oil to obtain an emulsion, whose final concentration varies depending on the type of petroleum (each petroleum has a distinct characteristic, resulting in dispersions with different properties and concentrations). Furthermore, the water conditions, such as salinity and pH value, can further be altered to provide a greater (or lesser) quantity of oil dispersed in the water, depending on the scenario observed in the field, and which is the object of mimicking.

The mixture of the method of the invention is made in this way to simulate the behavior of a separation vessel interface or electrostatic treater (or even desalter, in the case of petroleum refining), equipment that operate mainly by the action of gravity. Any type of emulsion that occurs and is more stable, considering the operating dynamics in a separation system, will result in process instability and oil carryover into the water. Furthermore, the invention aims at evaluating how the chemical products added in petroleum processing will influence the oil carryover to the aqueous phase. With this approach, it becomes possible to positively (or negatively) evaluate the carryover of a larger quantity of oil to the aqueous phase, considering the separation of water/oil present in a separation equipment. Such an evaluation is made by comparing the dispersion in the absence and presence of the chemical product targeted by the evaluation itself.

Finally, the document titled “TEOR DE ÓLEO E GRAXA (TOG), Método Gravimétrico—extração com hexano” (“OIL AND GREASE CONTENT (OGC), Gravimetric Method—extraction with hexane”) teaches a method for evaluating the OGC in an industrial effluent sample comprising acidification with HCl to pH 2, extraction 3 times with hexane and filtration, followed by evaporation of the solvent to determine the OGC.

This document discloses a gravimetric method for determining OGC (liquid-liquid extraction with hexane), which is only a tool possibly used in one of the steps of the method of the present invention. However, the invention is not restricted to such a method, and does not refer to the simple quantification of OGC.

Additionally, other processes are part of the present invention, such as, for example, the evaluation of the effect of products in reinjection or the evaluation of the impact of degreasers on oil carryover and water contamination. In the specific case of the reinjection, for example, the presence of products and additives may result in the carryover of a greater quantity of oil and rapid destabilization thereof, leading to rapid clogging of the filter, or to an effect of reducing interfacial tension, facilitating passage during the filtration.

It is noted that the documents of the state of the art do not simulate the water/oil separation process, considering separation vessels and related equipment. In this way, there is a need for the provision of methods for evaluating the impact of chemical products on the quality of produced water.

### SUMMARY OF THE INVENTION

The present invention aims at proposing, in a first embodiment, a method for evaluating the impact of chemical products on the OGC of produced water comprising the steps of:
(i) preparation of synthetic emulsions using oil mimicking produced water, produced water mimicked with the chemical product whose impact is to be evaluated and saline water with said chemical product (without oil);
(ii) measurement of the OGC of the synthetic emulsions prepared in step (i); and
(iii) evaluation of the obtained data to determine the impact of the presence of the chemical product.

In a second embodiment, the present invention proposes a method for evaluating the impact of chemical products on the reinjection of produced water comprising the following steps:
(I) preparation of synthetic emulsions using oil mimicking produced water and produced water mimicked with the chemical product whose impact is to be evaluated;
(II) measurement of the OGC of the synthetic emulsions prepared in step (I);
(III) measurement of the filtration time and the mass of oil retained in the membrane; and
(IV) evaluation of the obtained data to determine the impact of the presence of the chemical product.

### DETAILED DESCRIPTION OF THE INVENTION

In a first embodiment, the present invention refers to a method for evaluating the impact of chemical products on the OGC of produced water comprising the steps of:
(i) preparation of synthetic emulsions using oil, mimicking produced water, produced water mimicked with the chemical product whose impact is to be evaluated and saline water with said chemical product (without oil);
(ii) measurement of the OGC of the synthetic emulsions prepared in step (i); and
(iii) evaluation of the obtained data to determine the impact of the presence of the chemical product.

The synthetic emulsions of step (i) must be prepared simultaneously to ensure the repeatability and reproducibility of the results. In addition, they must be prepared in triplicate.

In step (i), the mimetic emulsion of produced water is prepared from the dispersion of an oil in water with salts and pH ranging from 5 to 12. In particular, the oil is a petroleum sample. In particular, the salts may be, but are not limited to: NaCl, KCl, CaCl₂, MgCl₂, BaCl₂, SrCl₂, NaHCO₃ and/or CaHCO₃. Preferably, the salt used is NaCl. In particular, the pH of the aqueous phase ranges from 7 to 12, preferably from 7 to 10, even more preferably the pH of the aqueous phase is 8.

In step (i), the water emulsion with the product to be evaluated is prepared from the dispersion of said product in water with salts. In particular, the salts may be, but are not limited to: NaCl, KCl, CaCl₂, MgCl₂, BaCl₂, SrCl₂, NaHCO₃ and/or CaHCO₃. In a preferred embodiment, the salt used is NaCl.

In step (i), the mimetic emulsion of produced water with the product whose impact is to be evaluated is prepared as described in paragraph [0036], with the addition of said product together with the addition of the oil.

In a preferred embodiment, in step (i), the mimetic emulsion of produced water or the mimetic emulsion of produced water with a chemical product is prepared from the steps of: a) addition of NaCl saline solution in a concentration of 20 to 100 g/L, more preferably 35 g/L; b) adjustment of the pH to a range of 5 to 12, more preferably 7 to 10, even more preferably 8, with a base; c) addition of the oil sample and/or the chemical product under stirring; d) rest for 1 to 4 hours, more preferably 2 hours; and e) drainage of the aqueous phase.

In a more preferred embodiment, in step (i), the base of step b) of the emulsion preparation process is NaOH, NH₄OH or Ca(OH)₂. In an even more preferred embodiment, the base is NaOH.

Step (ii) can be performed by methods known in the art for evaluating the content of oils and greases, such as, but not limited to, gravimetric method, infrared, fluorescence, ultrasound, photometry, ultraviolet, gas chromatography. In a preferred embodiment, the method used in step (ii) is the gravimetric method.

In an even more preferred embodiment, step (ii) is performed from the following steps: a) acidifying the aqueous phase drained in step (i) to pH 2; b) extracting with an organic solvent; c) draining the aqueous phase; d) filtering the organic phase with sodium sulfate; e) collecting the organic phase; f) repeating steps b) to e) twice; g) evaporating the solvent; and h) weighing the dry extract.

In step (ii), the organic solvent used in step b) may be, but is not limited to: hexane, cyclohexane, chloroform, dichloromethane or toluene. In an even more preferred embodiment, in step (ii), the solvent used in step b) is n-hexane.

In step (iii), to determine the impact of the chemical product analyzed, the OGC masses obtained in step (ii) are evaluated, and differences greater than 30% between the values are considered relevant. In this way, when the OGC of the emulsion with the chemical product is equal to or lower (considering the acceptable variation of 30%) than that of the mimetic emulsion of produced water without the chemical product, it is considered that the product has no impact on the OGC. On the other hand, when the OGC of the emulsion with the chemical product is higher (considering the acceptable variation of 30%) than that of the mimetic emulsion of produced water without the chemical product, the product has an impact on the OGC.

In an optional embodiment, the method of the invention comprises a step (iv) of selecting a chemical product, the product being selected if it presents a value lower or up to 30% higher (i.e., considered equal within the scope of the present methods) than the value presented for the mimetic emulsion of produced water.

In a second embodiment, the present invention refers to a method for evaluating the impact of chemical products on the reinjection of produced water comprising the steps of:
(I) preparation of synthetic emulsions using oil mimicking produced water and produced water mimicked with the chemical product whose impact is to be evaluated;
(II) measurement of the OGC of the synthetic emulsions prepared in step (I);
(III) measurement of filtration time and mass of oil retained in the membrane; and
(IV) evaluation of the obtained data to determine the impact of the presence of the chemical product.

In step (I), two sets of dispersions are prepared in triplicate, with one set of samples submitted to step (II) and the other to step (III). Steps (II) and (III) are performed independently of each other and are not sequential steps.

In step (I), the mimetic emulsion of produced water and the mimetic emulsion of produced water with the product whose impact is to be evaluated are prepared as described in paragraphs [0036], [0038], and [0040].

Step (II) is performed as described in paragraph [0041]. The purpose of step (II) is to determine the OGC for use as a reference in the interpretation of data in step (IV), being an additional criterion for the identification of products with less impact and, therefore, more suitable for use in the evaluated processing of petroleum.

In a more preferred embodiment, step (II) is carried out from the following steps: a) acidifying the aqueous phase drained in step (I) to pH 2; b) extracting with an organic solvent; c) draining the aqueous phase; d) filtering the organic phase with sodium sulfate; e) collecting the organic phase; and f) repeating steps b) to e) twice; g) evaporating the solvent; and h) weighing the dry extract.

In step (II), the organic solvent used in step b) may be, but is not limited to: hexane, cyclohexane, chloroform, dichloromethane or toluene. In an even more preferred embodiment, in step (II), the solvent used in step b) is n-hexane.

Step (III) comprises the steps of a) filtering the aqueous phase obtained in step (I) in a previously tared membrane system of mixed cellulose ester, cellulose acetate, glass fiber or nylon, preferably mixed cellulose ester; b) recording the filtration time; and c) after filtration, drying and recording the mass of the dry membrane.

In particular, the filtration time can be recorded with the aid of a timer.

In step (IV), the filtration times of step b) are compared. The shorter the filtration time of the mimetic emulsion of produced water with the chemical product compared to the mimetic emulsion of produced water, the lower the impact of the chemical product. Furthermore, the mass of oil retained and the OGC value obtained in step (II) are taken into account to identify more suitable products.

In an optional embodiment, the method of the invention comprises a step (V) of selecting a chemical product, the product being selected if f it presents a filtration time shorter than that of the mimetic emulsion of produced water and does not considerably increase the OGC and/or the mass retained in the membrane. A considerable increase is an increase from 50%.

In the context of the present invention, the chemical product to be evaluated may be, but is not limited to: a demulsifier, an emulsifier, a degreaser, an antifoam agent, a scale inhibitor, a corrosion inhibitor, a friction inhibitor, an H₂S scavenger, a biocide, polyelectrolytes, an O₂ scavenger, a foam-generating liquid or cleaning solvents.

The methods of the present invention allow the selection of chemical products with the lowest impact on the quality of the produced water for petroleum processing. Thus, the present invention allows a significant reduction in the impact of chemical products on OGC, with a consequent reduction in the occurrence and fines for non-compliance. Furthermore, the methods allow the evaluation of the impact on reinjection processes, allowing the identification of products that facilitate the filtration process, with potential application in the field to reduce the formation of damage, or, in the case of products with a negative impact, to interrupt or avoid their use.

In this way, the present invention presents the advantages of reducing the occurrence of irregularities in the OGC of discarded produced water and of reducing the number of interventions in reinjection wells due to the formation of damage.

In the context of the present invention, “emulsions” and “dispersions”, as they refer to the samples used in the methods described herein, should be understood as synonyms, referring to the mixtures of water (saline) with oils and/or the chemical products evaluated.

In the context of the present invention, “produced water” refers to the water that is co-produced together with petroleum and/or natural gas during the extraction of these resources from subterrain reservoirs, which may originate from different sources, including water naturally present in petroleum and gas reservoirs, as well as water that is injected during secondary or tertiary recovery processes.

In the context of the present invention, the “mimetic emulsion of produced water” or “emulsion of mimicked produced water” refers to a sample containing the aqueous phase of a mixture of salinized water with oil, prepared as described in the present disclosure.

In the context of the present invention, the “mimetic emulsion of produced water with product”, “emulsion of produced water mimicked with product”, “emulsion of produced water mimicked chemical product” or “mimetic emulsion of produced water with chemical product” refers to a sample containing the aqueous phase of a mixture of salinized water with oil and a chemical product whose impact is to be evaluated, prepared as described in the present disclosure.

In order to demonstrate its potential, the present invention will be described in more detail in terms of the embodied examples. It should be emphasized that the following description is only intended to elucidate the understanding of the proposed invention and to disclose, in more detail, the embodiment of the invention without limiting it to the same. In this way, variables similar to the examples are also within the scope of the invention.

### Example 1: Evaluation of the Impact of Biocides and Scale Inhibitors on OGC

### 1.1: Preparation of the Dispersions

Three emulsions/dispersions were prepared in triplicate, as described below.

### 1.1.1. Dispersion of the Oil in Water (Mimetic Emulsion of Produced Water)

In a separatory funnel, 500 ml of a 35 g/L NaCl saline solution were added, with pH adjusted to 8, with an aqueous NaOH solution of 1 molar concentration. Then, 5 mL of sample of petroleum A were added and the system was stirred for 5 minutes. After resting for 2 hours, the aqueous phase was drained into a vial and set aside.

Petroleum A has a total acidity index of 1.6 mg/kg KOH; asphaltene content of 4.5%, SAP (saturated, aromatic and polar) analysis equal to 37.1%, 32.6% and 30.3% respectively, and density 944.8 kg/m³.

### 1.1.2. Dispersion of the Chemical Product in Water

In a separatory funnel, 500 ml of saline solution 35 g/L of NaCl were added. Then, 1000 μL of the chemical product were added and the system was stirred for 5 minutes. After resting for 2 hours, the aqueous phase was drained into a flask and set aside.

The chemical products used were scale inhibitors and biocides, as indicated in the table below.

TABLE 1
Chemical Products Evaluated in Example 1

| | |
|---|---|
| Inhibitor A | Phosphorus-containing polymers (polyamino |
| | polyether methylene phosphonic acid) |
| | Solvscale ® OG 1173 B |
| Inhibitor B | Phosphorus-containing polymers (polyamino |
| | polyether methylene phosphonic acid) |
| | Scaletreat ® 15052 |
| Biocide A | Tetrakis methyl hydroxide phosphonic sulfate |
| | (THPS) - Trade name: Biotreat 4682 |
| Biocide B | Tetrakis methyl hydroxide phosphonic sulfate |
| | (THPS) - Trade name: 10168 NR |
| Biocide C | Tetrakis methyl hydroxide phosphonic sulfate |
| | (THPS) - Trade name: Dorf OG 304 B |

### 1.1.3. Dispersion of the Oil and Chemical Product in Water (Mimetic Emulsion of Produced Water With Chemical Product)

In a separatory funnel, 500 ml of 35 g/L NaCl saline solution, with pH adjusted to 8, with NaOH aqueous solution of 1 molar concentration were added. Then, 5 mL of petroleum sample and 1000 μL of the chemical product were added and the system was stirred for 5 minutes. After resting for 2 hours, the aqueous phase was drained into a vial and set aside.

The petroleum and chemical products used are those indicated in items 1.1.1 and 1.1.2 above.

### 1.2: OGC Evaluation

The aqueous phases separated in the previous step were acidified with 37% hydrochloric acid to pH 2. 10 mL of n-hexane were added to the aqueous phase and the vial was stirred to perform the extraction. The aqueous phase was drained after 5 min of resting. The organic fraction was filtered through a bed of sodium sulfate and collected in a previously weighed Erlenmeyer flask. The extraction with 10 ml of hexane was repeated twice, totaling approximately 30 mL of hexane extract. The final extract was evaporated under a nitrogen jet and weighed.

### 1.3: Results and Evaluation

Table 2 below shows the results and interpretations from the evaluation of the samples in item 1.1.

TABLE 2
Evaluation of the impact on the oil and grease content
(OGC) of the emulsions prepared in the presence
of different scale inhibitors and biocides

| | OGC | |
|---|---|---|
| Emulsion | (mg/L) | Interpretation |
| Petroleum A | 255 | Original emulsion. |
| Scale | 26 | No impact of the chemical product |
| inhibitor A | | on the increase in OGC was |
| (blank) | | observed. The final concentration |
| Petroleum A + | 226 | of the mixture is very similar to |
| Inhibitor A | | the emulsion with only oil. |
| Scale | N.D. | No impact of the chemical product |
| inhibitor B | | was observed. The concentration of |
| (blank) | | the mixture is even lower when |
| Petroleum A + | 119 | compared to the emulsion with only |
| Inhibitor B | | oil. |
| Biocide A | <10 | Potential impact of the chemical |
| (blank) | | product on the amount of dispersed |
| Petroleum A + | 2438 | oil (increase in the final OGC). |
| Biocide A | | |
| Biocide B | 17 | No impact of the chemical product |
| (blank) | | input on the increase in OGC was |
| Petroleum A + | 244 | observed. The final concentration |
| Biocide B | | of the mixture is very similar to the |
| | | emulsion with only oil. |
| Biocide C | <10 | No impact of the chemical product |
| (blank) | | input on the increase in OGC was |
| Petroleum A + | 238 | observed. The final concentration |
| Biocide C | | of the mixture is very similar to the |
| | | emulsion with only oil. |

N.D. = not detected.

For scale inhibitor A, biocide B and biocide C, it is observed that there is no significant difference in the resulting OGC when compared to the original emulsion. In the case of scale inhibitor B, a reduction in OGC is even observed in the presence of the product. As for biocide A, a significant increase in OGC is observed in the case of this product, and its use is not recommended when water is discarded.

### Example 2: Evaluation of the Impact of Degreasers on OGC

The samples used in Example 2 were prepared as indicated in item 1.1 of Example 1, using petroleum B and degreasers A and B, and the OGC was determined according to the method indicated in item 1.2 of Example 1 above.

Petroleum B has a total acidity index of 0.4 mg/kg KOH; asphaltene content of 2.4%, SAP (saturated, aromatic and polar) analysis equal to 47.18, 29.9% and 22.7% respectively, density 897.8 kg/m³.

Degreasers A and B are indicated in the table below.

TABLE 3
Chemical Products Evaluated in Example 2

| | |
|---|---|
| Degreaser | Surfactant polymers containing carbonyl groups |
| A | Roxil N (produced by Archem ®) |
| Degreaser | Surfactant polymers containing carbonyl groups |
| B | Versol Plus Eco |

Table 4 below shows the results and interpretations from the OGC evaluation of the samples from Example 2.

TABLE 4
Evaluation of the impact on the oil and grease content (OGC) of
the emulsions prepared in the presence of different degreasers

| | OGC | |
|---|---|---|
| Emulsion | (mg/L) | Interpretation |
| Petroleum B | 14 | Original emulsion. |
| Degreaser A | 7 | Potential impact of the chemical |
| (blank) | | product on the amount of dispersed |
| Petroleum B + | 506 | oil (increase in the final OGC). |
| Degreaser A | | |
| Degreaser B | N.D. | Potential impact of the chemical |
| (blank) | | product on the amount of dispersed |
| Petroleum B + | 132 | oil (increase in the final OGC). |
| Degreaser B | | |

N.D. = not detected.

The function of degreasers is to remove oil from equipment, and it is important that they dissolve the oil as efficiently as possible. There is a concern, when using this product, that OGC may increase if the fluids obtained after cleaning are aligned for the process. By applying the approach described herein, it was possible to select a new degreaser (B) that, when compared to the previous one (A), has a 4 times smaller impact on OGC.

### Example 3: Evaluation of the Impact of Emulsifiers on the Quality of Water Produced for Reinjection

### 3.1 Preparation of the Samples

Two sets of emulsions/dispersions were prepared in triplicate, as described below.

### 3.1.1 Dispersion of the Oil in Water (Mimetic Emulsion of Produced Water)

In a separatory funnel, 500 ml of a 35 g/L NaCl saline solution, with pH adjusted to 8, with an aqueous NaOH solution of 1 molar concentration were added. Then, 5 mL of a sample of petroleum C, petroleum D or petroleum E were added and the system was stirred for 5 minutes. After resting for 2 hours, the aqueous phase was drained into a vial and set aside.

Petroleum C has a total acidity index of 0.7 mg/kg KOH; asphaltene content of 1.5%, SAP (saturated, aromatic and polar) analysis equal to 46.4%, 30.6% and 21.5% respectively, density 917.8 kg/m³.

Petroleum D has a total acidity index of 3.0 mg/kg KOH; asphaltene content of 1.3%, SAP (saturated, aromatic and polar) analysis equal to 41.78, 28.7% and 30.6% respectively, density 947.9 kg/m³.

Petroleum E has a total acidity index of 6.7 mg/kg KOH; asphaltene content of 2.5%, SAP (saturated, aromatic and polar) analysis equal to 46.8%, 29.8% and 31.4% respectively, density 911.4 kg/m³.

### 3.1.2 Dispersion of the Oil and Chemical Product in Water (Mimetic Emulsion of Produced Water With Chemical Product)

In a separatory funnel, 500 ml of 35 g/L NaCl saline solution, with pH adjusted to 8, with an aqueous NaOH solution of 1 molar concentration were added. Then, 5 mL of petroleum sample and 1000 μL of the chemical product were added and the system was stirred for 5 minutes. After resting for 2 hours, the aqueous phase was drained into a vial and set aside.

The chemical products used were emulsifiers, indicated in the table below.

TABLE 5
Chemical Products Evaluated in Example 3

| | |
|---|---|
| Emulsifier 1 | Ethylene oxide and propylene copolymer |
| | Ultrawet ® 70 |
| Emulsifier 2 | Polyethylene glycol oleate |
| | Liovac ® 4269 |

### 3.2 OGC Evaluation

The OGC was measured according to the methodology presented in Example 1, item 1.2.

### 3.3 Injection Time Evaluation

The total volume of each sample prepared according to item 3.1 (500 mL) was filtered using a system with a previously tared cellulose acetate membrane (47 mm wide and 0.45 micron pore diameter). The filtration time was measured with the aid of a timer and recorded. After filtration, the membrane was dried and the corresponding mass noted.

### 3.4 Results and Evaluation

Table 6 below shows the results and interpretations from the evaluation of the samples in item 3.1 according to the method in item 3.3.

TABLE 6
Evaluation of the impact on reinjection and OGC of
emulsions prepared in the presence of emulsifiers

| | Filtered | Retained | |
|---|---|---|---|
| | Volume | Mass | Time |
| Emulsion | (mL) | (mg) | (min) |
| Petroleum C | 490 | 6.5 | 65 |
| Petroleum C with | 500 | 8.4 | 14 |
| Emulsifier 1 | | | |
| Petroleum C with | 500 | 11.4 | 50 |
| Emulsifier 2 | | | |
| Petroleum D | 495 | 6.9 | 97 |
| Petroleum D with | 500 | 13.1 | 58 |
| Emulsifier 1 | | | |
| Petroleum D with | 495 | 17.7 | 68 |
| Product 2 | | | |
| Petroleum E | 495 | 11.2 | 107 |
| Petroleum E with | 500 | 14.5 | 49 |
| Emulsifier 1 | | | |
| Petroleum E with | 505 | 17.4 | 31 |
| Emulsifier 2 | | | |

There is a significant difficulty involving produced water reinjection processes due to the residual OGC present. The oil present ends up forming damage (clogging) at the well/reservoir interface. The formation of this damage results in an increase in injection pressure. This pressure cannot be increased indefinitely, with the risk of fracturing the reservoir. Therefore, there is a search for solutions that minimize the formation of this damage.

It was observed that, for the samples evaluated, although a greater mass of oil was retained, the filtration time was much shorter. This indicates that the products are acting efficiently in reducing interfacial tension, facilitating the flow. This is very positive for reinjection in the short and medium term. However, there is a greater separation of the oil, still requiring interventions to remove the same.

1. A method for evaluating an impact of chemical products on OGC of produced water, comprising the steps of:
(i) preparation of synthetic emulsions using oil mimicking produced water, produced water mimicked with a chemical product whose impact is to be evaluated and saline water with said chemical product (without oil);
(ii) measurement of the OGC of the synthetic emulsions prepared in step (i);
(iii) evaluation of obtained data.

2. The method according to claim 1, wherein:
the mimetic emulsion of produced water is prepared from dispersion of an oil in water with salts and a pH of 5 to 12;
the water emulsion with the product to be evaluated is prepared from dispersion of said product in water with salts; and
the mimetic emulsion of produced water with the chemical product is prepared from dispersion of an oil and said chemical product in water with salts and a pH of 5 to 12.

3. The method according to claim 2, wherein:
the oil is a petroleum sample;
the salts are NaCl, KCl, CaCl₂, MgCl₂, BaCl₂, SrCl₂, NaHCO₃ and/or CaHCO₃; and/or
the pH of the aqueous phase ranges from 7 to 12.

4. The method according to claim 3, wherein the salt is NaCl.

5. The method according to claim 3, wherein the pH of the aqueous phase is 8.

6. The method according to claim 1, wherein in step (i), the mimetic emulsions of produced water and produced water with chemical product are prepared from the steps of:
a) addition of NaCl saline solution at a concentration of 20 to 100 g/L;
b) adjustment of the pH to a range of 7 to 10 with a base;
c) addition of the oil sample and/or the chemical product under stirring;
d) rest for 1 to 4 hours; and
e) drainage of the aqueous phase.

7. The method according to claim 6, wherein the NaCl saline solution is added at 35 g/L, the base is NaOH, the pH is adjusted to 8 and the rest is for 2 hours.

8. The method according to claim 1, wherein step (ii) comprises the steps of:
a) acidifying the aqueous phase drained in step (i) to pH 2;
b) extracting with an organic solvent;
c) draining the aqueous phase;
d) filtering the organic phase with sodium sulfate;
e) collecting the organic phase;
f) repeating steps b) to e) twice;
g) evaporating the solvent; and
h) weighing the dry extract.

9. The method according to claim 8, wherein the organic solvent is n-hexane.

10. The method according to claim 1, wherein in step (iii), products that lead to a OGC above 30% of that of the mimetic emulsion of produced water are considered to have an impact on the OGC.

11. The method according to claim 1, further comprising the step of:
(iv) selecting products, in which the products that have no impact on the OGC identified in step (iii) are selected for processing.

12. A method for evaluating an impact of chemical products on reinjection of produced water, comprising the steps of:
(I) preparation of synthetic emulsions using oil mimicking produced water and produced water mimicked with a chemical product whose impact is to be evaluated;
(II) measurement of OGC of the synthetic emulsions prepared in step (I);
(III) measurement of filtration time and mass of oil retained in a membrane; and
(IV) evaluation of obtained data.

13. The method according to claim 12, wherein:
the mimetic emulsion of produced water is prepared from dispersion of an oil in water with salts and a pH of 5 to 12; and
the mimetic emulsion of produced water with the chemical product is prepared from dispersion of an oil and said chemical product in water with salts and a pH of 5 to 12.

14. The method according to claim 13, wherein:
the oil is a petroleum sample;
the salts are NaCl, KCl, CaCl₂, MgCl₂, BaCl₂, SrCl₂, NaHCO₃ and/or CaHCO₃; and/or
the pH of the aqueous phase ranges from 7 to 12.

15. The method according to claim 14, wherein the salt is NaCl.

16. The method according to claim 14, wherein the pH of the aqueous phase is 8.

17. The method according to claim 12, wherein in step (I), the mimetic emulsions of produced water and produced water with chemical product are prepared from the steps of:
a) addition of NaCl saline solution at a concentration of 20 to 100 g/L;
b) adjustment of the pH to a range of 7 to 10 with a base;
c) addition of the oil sample and/or the chemical product under stirring;
d) rest for 1 to 4 hours; and
e) drainage of the aqueous phase;
optionally wherein the NaCl saline solution is added at 35 g/L, the base is NaOH, the pH is adjusted to 8 and the rest is for 2 hours.

18. The method according to claim 12, wherein the OGC measurement of step (II) comprises the steps of:
a) acidifying the aqueous phase drained in step (i) to pH 2;
b) extracting with an organic solvent;
c) draining the aqueous phase;
d) filtering the organic phase with sodium sulfate;
e) collecting the organic phase;
f) repeating steps b) to e) twice;
g) evaporating the solvent; and
h) weighing the dry extract;
optionally wherein the organic solvent is n-hexane.

19. The method according to claim 12, wherein step (III) comprises the steps of:
a) filtering the aqueous phase obtained in step (I) in a previously tared membrane system of mixed cellulose ester, cellulose acetate, glass fiber or nylon;
b) recording the filtration time; and
c) after filtration, drying and recording the mass of the dry membrane;
optionally wherein the membrane is a mixed cellulose ester membrane.

20. (canceled)

21. The method according to claim 12, wherein in step (IV), the filtration times of step b) and step (III) are compared to identify an increase or a decrease in the filtration time, wherein decrease in the filtration time indicates a lower impact.
